(19) United States
(12) Patent Application Publication
(10) Pub. No.: US 2008/0161839 A1
(43) Pub. Date: Jul. 3, 2008

(54) Device and Method for Coronary Artery Bypass Procedure

(75) Inventor: Alon Shalev, RaAnana (IL)

Correspondence Address: Martin D. Moynihan;PRTSI, P.O. Box 16446, Arlington, VA, 22215, US

(73) Assignee: Nicast Ltd., Lod (IL)

(21) Appl. No.: 11/795,913

(22) PCT Filed: Jan. 25, 2006
(86) PCT No.: PCT/IL06/00104

§ 371 Date: Jul. 24, 2007

Related U.S. Application Data
(60) Provisional application No. 60/646,541, filed on Jan. 25, 2005.

(51) Int. Cl. A61B 17/08; A61F 2/06

(52) U.S. Cl. 606/153; 623/1.16

(57) ABSTRACT

A method of bypassing a coronary artery being at least partially occluded. The method comprises: using a branching graft for establishing direct fluid communication between three or more vascular locations. Specifically direct fluid communication is established between an upstream vascular location, a downstream vascular location and a distal vascular location. The distal vascular location is preferably selected on a distal artery or a distal portion of an artery to ensure that arterial blood flow in the distal artery generates sufficient pressure gradient in the branching graft to maintain the direct fluid communication.

### FIELD AND BACKGROUND OF THE INVENTION

The present invention relates to implantable devices, and, more particularly, to a device and method suitable for bypassing an occluded or partially occluded coronary artery.

Coronary arteries can become partially restricted (stenotic) or completely clogged (occluded) with plaque, thrombus or the like. Coronary artery disease remains the leading cause of morbidity and mortality in western world. Coronary artery disease is manifested in a number of ways. For example, disease of the coronary arteries can lead to insufficient blood flow resulting in the discomfort and risks of angina and ischemia. In severe cases, acute blockage of coronary blood flow can result in myocardial infarction, leading to immediate death or damage to the myocardial tissue.

A number of approaches have been developed for treating coronary artery disease. In less severe cases, it is often sufficient to treat the symptoms with pharmaceuticals and lifestyle modification to lessen the underlying causes of disease. In more severe cases, the coronary blockage(s) can often be treated endovascularly using techniques such as balloon angioplasty, atherectomy, laser ablation, stents, hot tip probes and the like.

In cases where pharmaceutical treatment and/or endovascular approaches have failed or are likely to fail, it is often necessary to perform a coronary artery bypass graft procedure using open surgical techniques. Depending upon the degree and number of coronary vessel occlusions, a single, double, triple, or even greater number of bypass procedures may be necessary.

In coronary artery bypass graft procedure the patient's sternum is opened and the chest is spread apart to provide access to the heart. A source of arterial blood is then connected to a coronary artery downstream from an occlusion while the patient is maintained under cardioplegia and is supported by cardiopulmonary bypass. The source of blood is often the left or right internal thoracic artery, and the target coronary artery can be the left anterior descending artery or any other coronary artery which might be narrowed or occluded.

Each bypass is accomplished by the surgical formation of a separate conduit from the aorta to the stenosed or obstructed coronary artery at a location distal to the diseased site. Typically, a suitable blood vessel is harvested from another part of the patient's body for use as a graft. The graft is used to create a new, uninterrupted channel between a blood source, such as the aorta, and the occluded coronary artery or arteries downstream from the arterial occlusion or occlusions.

A major obstacle has been the limited number of vessels that are available to serve as grafts. Potential grafts include the two saphenous veins of the lower extremities, the two internal thoracic arteries under the sternum and the single gastroepiploic artery in the upper abdomen. Thus, in general clinical practice, there are five vessels available to use in this procedure over the life of a particular patient. Once these “spare” vessels have been sacrificed, there is little or nothing that modern medicine can offer.

Attempts have been made to develop new procedures in which a single vessel is used to bypass multiple sites. A major drawback of this technique is that the physical stress (e.g., torsion) on the implanted graft is proportional to the number of bypasses for which it is being used. When the graft is used for many bypasses, the resulting physical stress is detrimental.

Attempts have also been made to use grafts from other species (xenografts), or other non-related humans (homografts). These attempts, however, have been largely unsuccessful.

Artificial vascular prostheses, such as those used for peripheral vascular bypass, vascular replacement and vascular access procedures, are well known and widely available in a variety of designs and configurations. Of particular interest are devices made of, or coated with, polymer materials which typically exhibit a microporous, open cell structure that in general allows healthy tissue growth and cell endothelization, thus contributing to the long term healing of the prostheses. Prostheses having sufficient porous structure tend to promote tissue ingrowth and cell endothelization along their inner surface.

A promising manufacturing technique of vascular prostheses is electro-capillary spinning also abbreviated to electrospinning. Electrospinning is a method for the manufacture of ultra-thin synthetic fibers which reduces the number of technological operations required in the manufacturing process and improves the product being manufactured in more than one way.

The process of electrospinning creates a fine stream or jet of liquid that upon proper evaporation of a solvent or liquid to solid transition state yields a nonwoven structure. The fine stream of liquid is produced by pulling a small amount of polymer solution through space by using electrical forces. More particularly, the electrospinning process involves the subjection of a liquefied substance, such as polymer, into an electric field, whereby the liquid is caused to produce fibers that are drawn by electric forces to an electrode, and are, in addition, subjected to a hardening procedure. In the case of liquid which is normally solid at room temperature, the hardening procedure may be mere cooling; however other procedures such as chemical hardening (polymerization) or evaporation of solvent may also be employed. The produced fibers are collected on a suitably located precipitation device and subsequently stripped from it. The sedimentation device is typically shaped in accordance with the desired geometry of the final product, which may be for example tubular, flat or even an arbitrarily shaped product.

The use of electrospinning for manufacturing or coating of vascular prostheses permits to obtain a wide range of fiber thickness (from tens of nanometers to tens of micrometers), achieves exceptional homogeneity, smoothness and desired porosity distribution along the coating thickness. When a graft is electrospinningly coated by a graft of a porous structure, the pores of the graft component are invaded by cellular tissues from the region of the artery surrounding the stent. Moreover, diversified polymers with various biochemical and physico-mechanical properties can be used in stent coating.

Nevertheless, there are several unresolved problems associated with traditional coronary artery bypass graft procedures, irrespectively of the type of graft used in the procedure. One such problem is the risk of kinking or collapsing of the graft under a variety of circumstances, such as when the graft is bent during the contraction of the surrounding muscle or tissue or when external pressure is applied to the graft when the graft recipient moves. Another problem is a post procedure obstruction of the graft due to neointimal proliferation or thrombus formation. The risk of post procedure obstruction is higher for small diameter vascular grafts (internal diameters less than about 6 mm) but is not negligible for larger diameter.

There is thus a widely recognized need for, and it would be highly advantageous to have device and method suitable for bypassing an occluded or partially occluded coronary artery, devoid of the above limitations.

### SUMMARY OF THE INVENTION

According to one aspect of the present invention there is provided a method of bypassing a coronary artery being at least partially occluded. The method comprises: using a branching graft for establishing direct fluid communication between an upstream vascular location being upstream the occlusion in the artery, a downstream vascular location being downstream the occlusion, and a distal vascular location. The distal vascular location is preferably selected on a distal artery or a distal portion of an artery to ensure that arterial blood flow in the distal artery generates sufficient pressure gradient in the branching graft to maintain the direct fluid communication.

According to further features in preferred embodiments of the invention described below, the method further comprises establishing direct fluid communication between at least one additional downstream vascular location, and the above vascular locations.

According to still further features in the described preferred embodiments the branching graft comprises at least one harvested blood vessel.

According to still further features in the described preferred embodiments the branching graft comprises an artificial graft.

According to another aspect of the present invention there is provided an artificial branching graft for implantation in body vasculature during a coronary artery bypass procedure, comprising: a primary conduit, at least one secondary conduit branching from the primary conduit, and at least one unidirectional valve designed and constructed to ensure unidirectional flow within at least a portion of the primary conduit.

According to still further features in the described preferred embodiments at least a portion of the primary conduit has a generally oval cross-sectional shape.

According to still further features in the described preferred embodiments the primary conduit is connected to the distal vascular location at an acute angle defined relative to the arterial blood flow. This can be achieved, for example, by providing a branching graft in which at least one end of the primary conduit is bent (e.g., at an acute angle) with respect to a longitudinal axis of the primary conduit. According to still further features in the described preferred embodiments the bending of the primary conduit is characterized by an acute angle measured at a convex side of the bending. According to still further features in the described preferred embodiments the acute angle is smaller or equals 70 degrees.

According to still further features in the described preferred embodiments the primary conduit of the branching graft is characterized by a varying cross-sectional area.

According to still further features in the described preferred embodiments the varying cross-sectional area varies in a non-monotonic manner.

According to still further features in the described preferred embodiments the varying cross-sectional area varies in a monotonic manner.

According to still further features in the described preferred embodiments the varying cross-sectional area is larger at the upstream vascular location than at the distal vascular location.

According to still further features in the described preferred embodiments the varying cross-sectional area has a minimal value at location on the primary conduit being other than the ends of the primary conduit.

According to still further features in the described preferred embodiments at least one of the primary conduit and the secondary conduit(s) is a tubular structure of non-woven polymer fibers.

According to still further features in the described preferred embodiments the branching graft comprises at least one generally annular flexible support structure supporting at least one end of the primary conduit and/or the secondary conduit(s). According to still further features in the described preferred embodiments the annular support structure is an embedded annular support structure.

According to still further features in the described preferred embodiments the branching graft further comprises a tubular support structure extending along at least one of the primary conduit and/or the at least one secondary conduit. According to still further features in the described preferred embodiments the tubular support structure is embedded in the respective conduit. According to still further features in the described preferred embodiments at least one of the primary conduit and/or the secondary conduit(s) comprises a plurality of layers each layer of the plurality of layers being made of non-woven polymer fibers.

According to still further features in the described preferred embodiments at least one of the primary conduit and/or the secondary conduit(s) includes a pharmaceutical agent incorporated therein for delivery of the pharmaceutical agent into the body vasculature during or after implantation of the branching graft within the body vasculature.

According to still further features in the described preferred embodiments the distal vascular location is on the aorta. According to still further features in the described preferred embodiments the distal vascular location is on the descending aorta.

According to still further features in the described preferred embodiments the distal vascular location can be on any suitable artery having sufficient blood flow to generate the desired pressure gradient, including, without limitation, the aortic arch, an aortic branch, the brachiocephalic artery, the left carotid artery, the left subclavian artery.

According to still further features in the described preferred embodiments the upstream vascular location can be on any artery capable of supplying blood to the downstream vascular location, including, without limitation, the ascending aorta, the (left or right) coronary artery and an aortic branch (e.g., the left subclavian artery).

According to still further features in the described preferred embodiments the upstream vascular location can be on the myocardium or on any artery downstream the occlusion, including, without limitation, the coronary artery and the coronary artery.

The present invention successfully addresses the shortcomings of the presently known configurations by providing a method and an artificial graft which enjoy properties far exceeding the prior art, in particular when used in coronary artery bypass procedures.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawing. With specific reference now to the drawing in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawing making apparent to those skilled in the art how the several forms of the invention may be embodied in practice.

In the drawings:

FIG. 1 is a schematic illustration of the human heart and the major blood vessels;

FIG. 2 is a flowchart diagram of a method suitable for bypassing a coronary artery being at least partially occluded, according to various exemplary embodiments of the present invention;

FIGS. 3a-b are schematic illustration of the heart and a branching vascular graft which can be used in coronary artery bypass procedure, according to various exemplary embodiments of the present invention;

FIG. 4a is a schematic illustration of a portion of a primary conduit of the vascular graft, in preferred embodiments in which the diameter of the primary conduit increases towards the connection with a distal blood vessel;

FIGS. 4b-c are schematic illustrations of the primary conduit of the vascular graft, in preferred embodiments in which the primary conduit has a cross-sectional area which varies monotonically (FIG. 4b) or non-monotonically (FIG. 4c) along the primary conduit;

FIG. 5 is a schematic illustration of the graft in preferred embodiments in which the primary and/or secondary conduits include more than one layer of non-woven polymer fibers; and

FIGS. 6a-b are schematic illustration of an electrospinning apparatus (FIG. 6a) and a precipitation electrode (FIG. 6b), according to various exemplary embodiments of the present invention.

### DESCRIPTION OF THE PREFERRED EMBODIMENTS

The present embodiments comprise a method and blood vessel, which can be used in medical invasive procedures. Specifically, the present embodiments can be used in coronary artery bypass procedures.

The principles and operation of a vascular prosthesis according to the present embodiments may be better understood with reference to the drawings and accompanying descriptions.

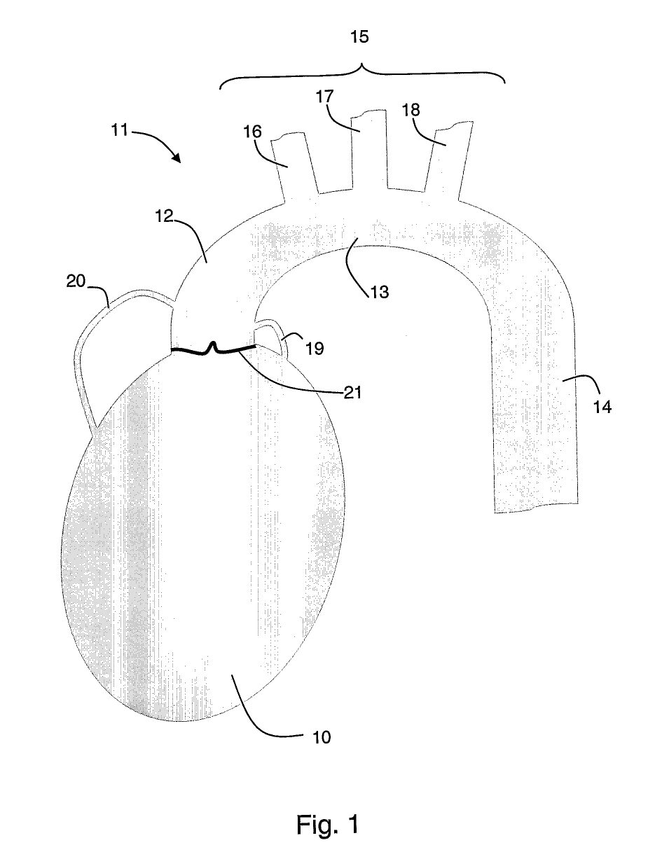

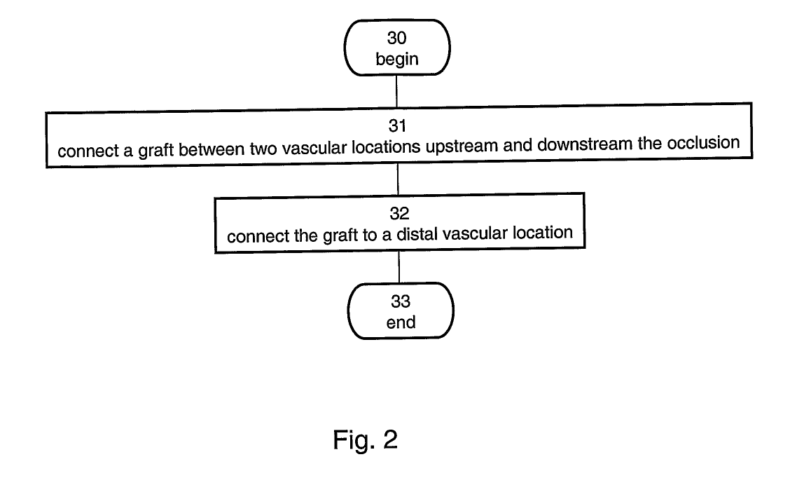

For purposes of better understanding the present invention, as illustrated in FIGS. 2-5 of the drawings, reference is first made to a schematic illustration of a human heart, shown in FIG. 1.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Referring now to the drawings, FIG. 1 is a schematic illustration of the human hart and the major blood vessels. One skilled in the art will recognize that several blood vessels and organs have been omitted from FIG. 1 for clarity of presentation. Shown in FIG. 1 are the heart mussel (myocardium) 10 and the thoracic aorta 11 which is commonly divided into the ascending aorta 12, the aortic arch 13 and the descending aorta 14. The abdominal part of the aorta is not shown in FIG. 1. Three major arteries, branching out of aortic arch 13 and conjointly referred to as the aortic branches 15, generally supply blood to the upper part of the body (head, upper limbs, neck and thorax). Aortic branches 15 include the brachiocephalic artery 16, the left carotid artery 17 and the left subclavian artery 18. The two main coronary arteries, the left coronary artery 19 and the right coronary artery 20, branch out from ascending aorta 12 to supply blood to heart mussel 10. Arteries 19 and 20 are smaller in size than aortic branches 15. Also shown in FIG. 1 is the aortic valve 21 which keeps blood from leaking back from ascending aorta 12 into the left ventricle.

When one or both of main coronary arteries 19 and 20 is occluded, e.g., due to a buildup of plaque, the blood flow to heart mussel 10 is reduced or totally stopped, leading to damage of the tissue in heart mussel 10. In a coronary artery bypass graft procedure, a graft, typically a harvested blood vessel, is traditionally connected to establish fluid communication between two vascular locations downstream and upstream the occlusion. The blood flow is redirected from the upstream vascular location through the graft and into the downstream vascular location, to thereby bypass the occlusion and renew the blood flow to heart mussel 10.

The downstream vascular location depends on the location of the occlusion. It can be on the main coronary artery (left 19 or the right 20 coronary artery), just below the occlusion, or on one the branches of the main coronary artery. The upstream vascular location depends on the type of graft used. Typically, when the saphenous vein is used, the upstream vascular location is on ascending aorta 12, and when the left internal thoracic artery is used, the upstream vascular location is on left subclavian artery 18. As stated in the Background section above, traditional coronary artery bypass graft procedures suffer from several limitations, including post procedure neointimal proliferation or thrombus formation in the implanted graft which may result in its obstruction.

In a search for improving the efficiency of coronary bypass grafting, the Inventor of the present invention has uncovered that the use of more than two vascular locations can significantly reduce the risk of post procedure obstruction. Generally, in a coronary artery bypass procedure performed according to the present embodiments, direct fluid communication is established between a vascular location upstream the occlusion, a vascular location downstream the occlusion, and a distal vascular location, which can be on any distal artery or a portion thereof, including, without limitation, descending aorta 14 or any one of the aortic branches 15. It was found by the present Inventor, that the fluid communication with the distal location can maintain sufficient blood flow between the other two locations (upstream and downstream the occlusion) for a prolonged period of time. As further described hereinbelow, due to the fluid communication with the distal location, low pressure regions are generated in the branching graft at an amount which is sufficient to reduce or prevent occlusion of the graft.

Referring now to the drawings, FIG. 2 is a flowchart diagram of a method suitable for bypassing a coronary artery being at least partially occluded, according to various exemplary embodiments of the present invention. It is to be understood that, unless otherwise defined, the method steps described hereinbelow can be executed either contemporaneously or sequentially in many combinations or orders of execution. Specifically, the ordering of the flowchart of FIG. 2 is not to be considered as limiting. For example, two or more method steps, appearing in the following description or in the flowchart of FIG. 2 in a particular order, can be executed in a different order (e.g., a reverse order) or substantially contemporaneously.

The method begins at step 30 and continues to steps 31-33 in which a branching graft is preferably connected to the two vascular locations, upstream and downstream the occlusion, and further connected to the distal vascular location to establish fluid communication between the three vascular locations.

The upstream vascular location can be any upstream vascular location commonly practiced during a conventional coronary artery bypass procedure, including, without limitation, a location on the ascending aorta or a location on the occluded or partially occluded coronary artery upstream the occlusion. Also contemplated are upstream vascular locations on one of the aortic branches.

The downstream vascular location depends on the location of the occlusion and can also be any downstream vascular location commonly practiced during a conventional coronary artery bypass procedure. Representative examples of suitable downstream vascular locations include, without limitation, a location on the occluded or partially occluded coronary artery downstream the occlusion, or a location on one of the branches of the coronary artery, e.g., the diagonal artery, the anterior descending artery, the posterior descending artery and the like.

The distal vascular location, which, as stated, can be on any distal artery or a portion thereof, is preferably selected such that arterial flow in the distal vascular location (e.g., blood flow through arteries 14, 16, 17 or 18, see FIG. 1) generates sufficient pressure gradient (with decreasing pressure in the direction of the distal vascular location) in the branching graft to maintain the direct fluid communication.

The method ends at step 33.

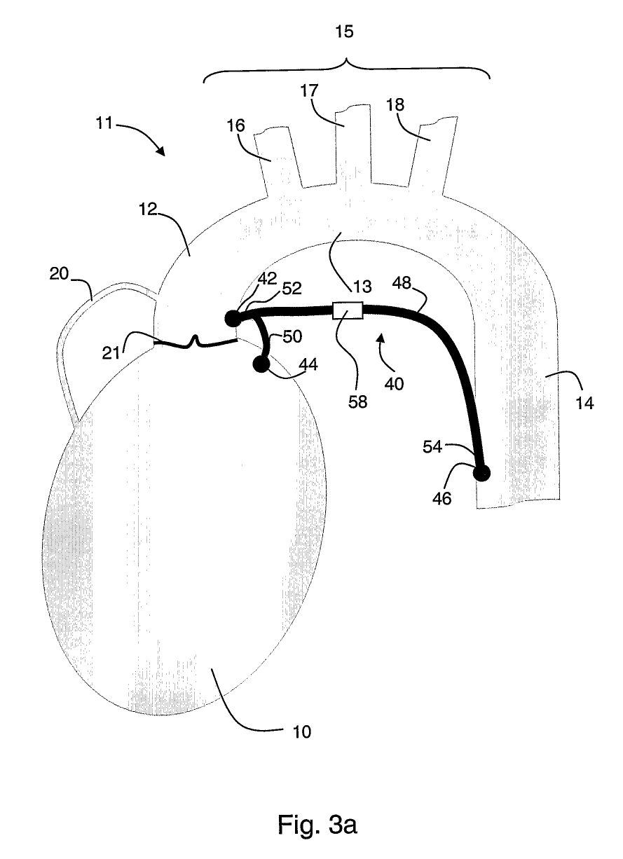

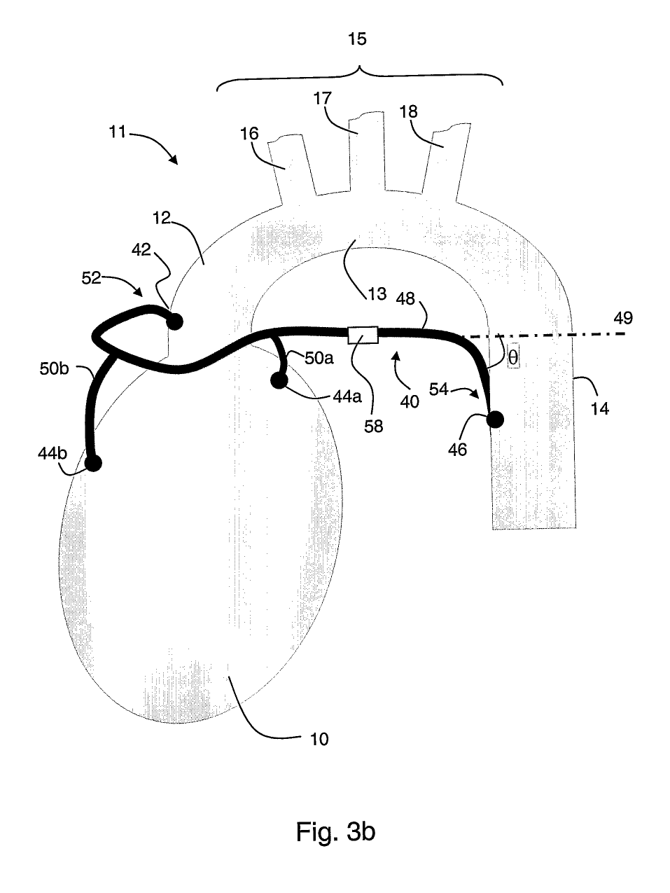

Reference is now made to FIGS. 3a-b, which are schematic illustration of heart 10 and a branching vascular graft 40 used in coronary artery bypass procedure, according to various exemplary embodiments of the present invention. Branching graft 40 comprises a primary conduit 48 having ends, generally designated by numerals 52 and 54, and one or more secondary conduits 50, branching from primary conduit 48. Graft 40 can comprises one or more harvested blood vessels or, more preferably, but not obligatorily, it can comprise an artificial graft.

During the coronary artery bypass procedure, end 52 is preferably connected to an upstream vascular location 42, and end 54 is preferably connected to a distal vascular location 46. In the representative illustration shown in FIGS. 3a-b, location 42 is on ascending aorta 12, and location 46 is on descending aorta 14. However, this need not necessarily be the case, since, as stated, it may not be necessary for the upstream vascular location to be on ascending aorta 12, or for distal vascular location 46 to be on descending aorta 14.

According to a preferred embodiment of the present invention at least a portion of primary conduit 48 has a generally oval cross-sectional shape. End 52 and/or end 54 can be bent with respect to the longitudinal axis 49 of primary conduit 48 to facilitate its connection to the vascular locations as further detailed hereinbelow. Preferably, but not obligatorily, end 54 is bended. The bending of end 54 is advantageous because it facilitates the proper connection to location 46 and prevents blood flow in the opposite direction within conduit 48. The bending also facilitates the generation the aforementioned pressure gradient in conduit 48 (with decreasing pressure in the direction of end 54). The bending of primary conduit 48 is preferably characterized by an acute angle, θ, measured at the convex side of the bending. In various exemplary embodiments of the invention θ is below 70°.

Graft 40 can comprise one, two or more secondary conduits 50 which serve for connecting graft 40 to one or more downstream vascular locations, depending on the required number of bypasses to the particular patient. For example, as illustrated in FIG. 3a, graft 40 can comprise one secondary conduit 50, in which case graft 40 is used for supplying blood to a single downstream vascular location 44. This embodiment is particularly useful in coronary artery bypass procedure in which only an occlusion in one coronary artery (left coronary artery 19, in the present example) is bypassed. As will be appreciated by one of ordinary skill in the art, this embodiment is also useful for bypassing occlusion on arteries which branch from the main coronary arteries, in which case the downstream vascular location 44 is preferably on the occluded artery, downstream its occlusion.

In another preferred embodiment, illustrated in FIG. 3b, graft 40 comprises two secondary conduits 50a and 50b, in which case graft 40 is used for supplying blood to two downstream vascular locations 44a and 44b, respectively. This embodiment can be useful in procedures in which only occlusions in both coronary arteries are bypassed. Similarly to the above, this embodiment is also useful for bypassing occlusion on arteries which branch from the main coronary arteries, whereby locations 44a and 44b are preferably on the occluded arteries, downstream their occlusion.

The connections of graft 40 to locations 42, 44 and 46 are via anastomoses, typically end-to-side anastomoses, marked in FIGS. 3a-b as full circles at the respective locations. It is to be understood that although the representative illustration of FIGS. 3a-b show locations 44, 44a and 44b on the myocardium 10, this should not be considered as limiting. The downstream vascular locations can be on any artery being downstream the occlusion(s) being bypassed, as further detailed above. Additionally, it may not be necessary for all the anastomoses to be end-to-side anastomoses, because in some cases, as will be appreciated by the one ordinarily skilled in the art, it may be more convenient to form an end-to-end anastomosis or not to form an anastomosis at all. For example, when a portion of graft 40 is the left internal thoracic artery, only its distal side is harvested to form one free end, while the other side remains connected to left subclavian artery 18. In this case, there no need to create anastomosis on left subclavian artery 18. On the other hand, in some procedures it may be desired to connect a portion of graft 40 to the free end of the left internal thoracic artery via end-to-end anastomosis.

Irrespectively of the type of anastomosis used, once graft 40 is connected to all the locations fluid communication is established between upstream location 42, downstream location 44 and distal location 46. The blood flow in the location 46 (descending aorta 14, in the present example) generate, e.g., via the Bernoulli effect, sufficient pressure gradient in conduit 48 to maintain the fluid communication between locations 42 and 44. Thus, the present embodiments provide an effective suction mechanism which enhances the blood supply to the downstream location. Such mechanism reduces or prevents accumulation of plaque thrombus or the like in the flow path from the upstream location and the downstream location. This is because as a result of suction forces directed towards distal location 46, plaque buildup or thrombus are drawn away from the flow path and keep the flow path substantially devoid of obstructions.

According to a preferred embodiment of the present invention graft 40 comprises one or more unidirectional valves 58 which ensure that there is a unidirectional flow within primary conduit 48 or a portion thereof. The unidirectional flow is preferably towards distal location 46.

Valve 58 can be any unidirectional valve known in the art which can be used in vascular prostheses. For example, valve 58 may be formed of a rigid annulus and one or more leaflets pivotally mounted within the annulus and capable of assuming an open position when the blood flow is in one direction and a closed position when the blood flow is in the other direction. Such and other types of unidirectional valves suitable for use in the present embodiments are disclosed in many patents and patent applications, see, e.g., U.S. Pat. Nos. 5,824,061, 6,126,686, 6,676,699 and 5,609,626 and 5,123,919, the contents of which are hereby incorporated by reference.

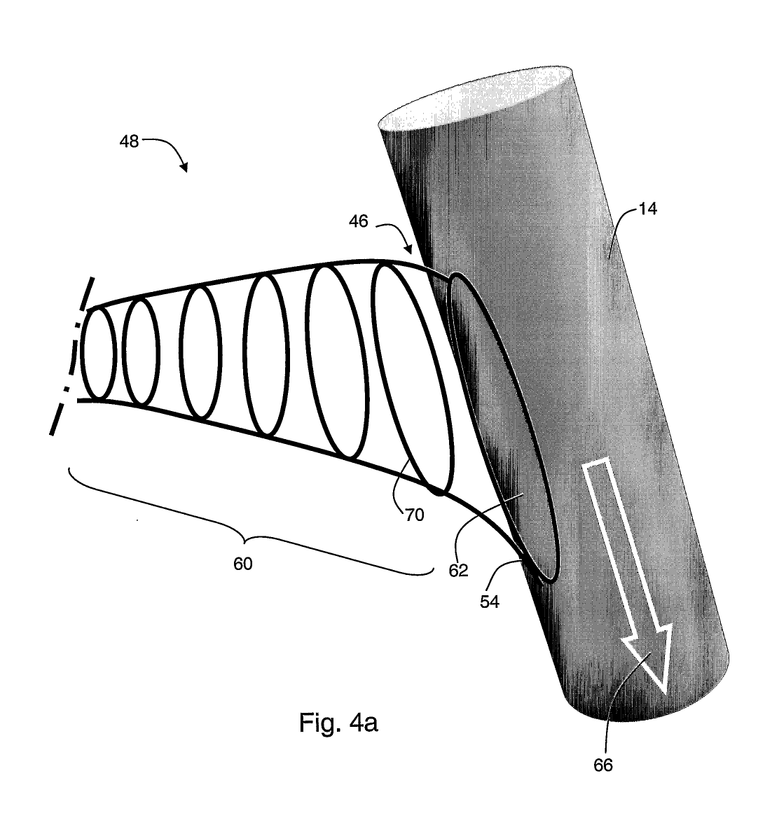

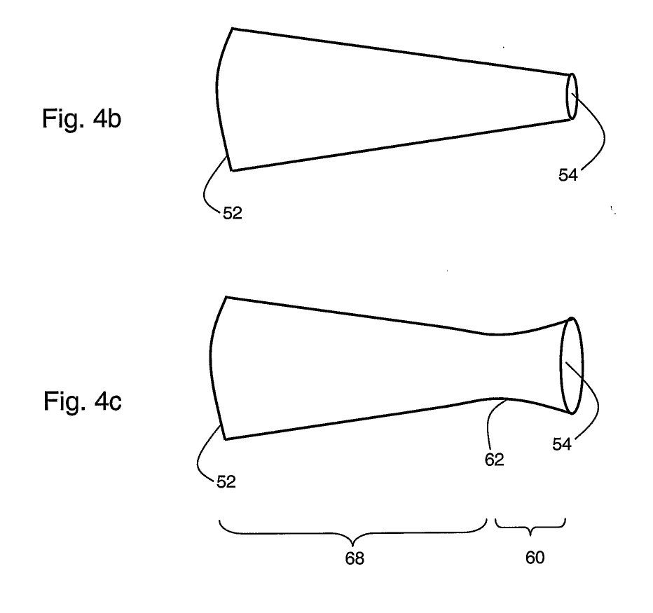

In various exemplary embodiments of the invention graft 40 comprises one or more generally annular flexible support structures 70 (not shown, see FIG. 4a) which supports one or more ends of conduits 48 and/or 50. Structure 70 can be embedded in the walls of the respective conduit or it can be attached externally or internally to the conduit. Structure 70 facilitates the connection of graft 40 to the various vascular locations. Annular support structure 70 can be any annular support structure known in the art (to this end see, e.g., WO 02/49535 ibid supra, and U.S. Pat. Nos. 5,984,973, 6,676,699 supra, 6,945,993, 6,949,120 and 6,939,373).

For example, annular structure 70 can be made of a metallic material such as, but not limited to, medical grade stainless steel, a cobalt alloy or a material exhibiting temperature-activated shape memory properties, such as Nitinol.

While reducing the present invention to practice it has be uncovered that a proper blood flow through graft 40 can be ensured by a judicious construction of the profile of the graft. In particular it was found that the shape of the profile of conduit 48 can maintain direct fluid communication between upstream location 42 and downstream location(s) 44.

Reference is now made to FIG. 4a which is a schematic illustration of a portion 60 of conduit 48 which includes end 54, according to various exemplary embodiments of the present invention. Also shown in FIG. 4 is a portion of the distal artery (descending aorta 14, in the present example) to which end 54 is connected via anastomosis 62. Blood flows through descending aorta 14 in the direction generally indicated by arrow 66. As shown in FIG. 4, conduit 48 is characterized by a varying cross-sectional area. In portion 60 of conduit 48 the cross-sectional area increases towards end 54 and anastomosis 62 of distal location 46.

Other preferred profiles of conduit 48 are schematically illustrated in FIG. 4b-c. In the preferred embodiment illustrated in FIG. 4b, the cross-sectional area of conduit 48 varies in a monotonic manner, whereby the varying cross-sectional area is larger at end 52 (near location 42, see FIGS. 3a-b) than at end 54 (near location 46). In the preferred embodiment illustrated in FIG. 4b, the cross-sectional area of conduit 48 varies in a non-monotonic manner, such that the cross-sectional area has a minimal value at a location 64 on conduit 48. Location 64 is preferably not at ends 52 or 54. Location 64 can be considered as separating between a portion 68 of conduit 48 located at the side of end 52 and portion 60 located at the side of end 54. Along portion 68, the cross-sectional area decreases, preferably monotonically, from a larger value at end 52 to a smaller value at location 64, and along portion 60, the cross-sectional area increases, preferably monotonically, from the smaller value at location 64 to a larger value at end 54.

The narrowing of conduit 48 facilitates the branching of the graft 40. In particular, the narrowing enables the connection between conduit 50, which is typically connected to a smaller blood vessel on the coronary vascular tree, and conduit 48 which is typically connected to the aorta or the aortic branches. The widening of conduit 48 facilitates the Bernoulli effect. The gradually increasing cross sectional surface area, results in a slower blood velocity within conduit 48. Flowing slowly through conduit 48, the blood arrives at end 54 where in intercepts with the relatively high average blood velocity in the aorta or the aortic branches. The Bernoulli effect thus takes place and a pressure gradient is formed with decreasing pressure towards end 54 of conduit 48. The pressure gradient results in suction of blood and debris from the connection between conduit 48 and 50 and reduces or prevent occlusion.

The length of each portion of conduit 48 may vary and depends on the total length of conduit 48. Without limiting the scope of the present invention to any specific dimension, the typical total length of conduit 48 is from about 15 cm to about 30 cm, the typical length of portions 60 and/or 68 is from about 0.5 cm to about 4 cm and the typical length of conduit(s) 50 is less than 15 cm. Other lengths are also contemplated.

As used herein the term “about” refers to ±10%.

Any one of primary conduit 48 and/or secondary conduit(s) 50 can be tubular structure of non-woven polymer fibers. The polymer fibers can be manufactured using any technique for forming non-woven fibers, such as, but not limited to, an electrospinning technique, a wet spinning technique, a dry spinning technique, a gel spinning technique, a dispersion spinning technique, a reaction spinning technique or a tack spinning technique.

Suitable electrospinning techniques are disclosed, e.g., in International Patent Application, Publication Nos. WO 2002/049535, WO 2002/049536, WO 2002/049536, WO 2002/049678, WO 2002/074189, WO 2002/074190, WO 2002/074191, WO 2005/032400 and WO 2005/065578, the contents of which are hereby incorporated by reference.

Other spinning techniques are disclosed, e.g., U.S. Pat. Nos. 3,737,508, 3,950,478, 3,996,321, 4,189,336, 4,402,900, 4,421,707, 4,431,602, 4,557,732, 4,643,657, 4,804,511, 5,002,474, 5,122,329, 5,387,387, 5,667,743, 6,248,273 and 6,252,031 the contents of which are hereby incorporated by reference.

A preferred technique for manufacturing branching graft suitable for the present embodiments is provided hereinunder.

The internal diameter of conduits 48 and 50 depend on the diameter of the arteries to which they are connected. Typically, the internal diameter is from about 1 mm to about 30 mm, more preferably from about 2 mm to about 20 mm, most preferably from about 2 mm to about 6 mm. When conduit 48 has a varying cross sectional area, its diameter at the location of minimal area is preferably from about 1 mm to about 10 mm and its diameter at the location of maximal area is preferably from about 10 mm to about 30 mm. Preferred wall thickness of the tubular structures is from about 0.1 mm to about 2 mm, more preferably from about 0.3 mm to about 1 mm, most preferably from about 0.5 mm to about 0.8 mm.

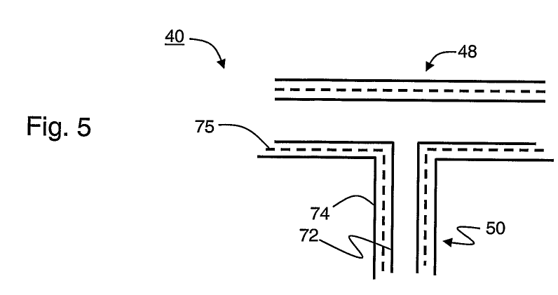

Reference is now made to FIG. 5, which is a schematic illustration of graft 40 in a preferred embodiment in which the primary and/or secondary conduits include more than one layer of non-woven polymer fibers. Two layers, a liner layer 72 and a cover layer 74, are illustrated in FIG. 5, but it is not intended to limit the scope of the present invention to any particular number of layers. Specifically, one or both conduits can include three or more layers of non-woven polymer fibers.

The advantage of using a plurality of layers is that with such configuration each layer can have different properties, such as porosity and/or mechanical strength, depending on its function. For example, liner layer 72, which typically serves as a sealing layer to prevent bleeding, can be manufactured substantially as a smooth surface with relatively low porosity. Layer 72 thus prevents bleeding and preclotting. In addition, throughout the life of the vascular graft, layer 72 ensures antithrombogenic properties and efficient endothelization of the inner surface of the vascular graft. A typical thickness of layer 72 is from about 40 μm to about 80 μm.

The requisite mechanical properties (high compliance, high breaking strength, etc.) of the vascular graft of the present embodiments are typically provided by the outer layers (e.g., cover layer 74). Thus, according to a preferred embodiment of the present invention the thickness of layer 74 is larger than the thickness of layer 72. A typical thickness of layer 74 is from about 50 μm to about 1000 μm.

Additionally, the porosity of layer 74 is preferably larger than the porosity of layer 72. A porous structure is known to promote ingrowth of surrounding tissues, which is extremely important for fast integration and long-term patency of the vascular graft. When the vascular graft comprises more than two layers, the porosity of the intermediate layers can differ from the porosities of the inner and outer layers. For example, the porosity of the layers can be a decreasing function of a distance of the layer from the center of the vascular graft.

Drug delivery into the body vasculature can be performed during or after implantation of the graft. Hence, according to a preferred embodiment of the present invention, one or more of the layers of graft 40 incorporates a pharmaceutical agent for delivery of the pharmaceutical agent into the body vasculature during or after implantation of graft 40. The pharmaceutical agent and its concentration can be selected in accordance with the expected pathology. The incorporated pharmaceutical agent can be a medicament for treating a particular disorder, an imaging agent to enable post implantation imaging, and the like.

Representative examples for suitable medicaments include, without limitation, heparin, tridodecylmethylammonium-heparin, epothilone A, epothilone B, rotomycine, ticlopidine, dexamethasone and caumadin

Also contemplated are other pharmaceutical agents such as, but not limited to, antithrombotic, estrogens, corticosteroids, cytostatic, anticoagulant, vasodilator, antiplatelet, trombolytics, antimicrobials, antibiotics, antimitotics, antiproliferatives, antisecretory, nonsterodial antiflammentory, grow factor antagonists, free radical scavengers, antioxidants, radiopaque agents, immunosuppressive and radio-labeled agents.

Conduits 48 and 50 can be made of any known biocompatible polymer. In the layers which incorporate pharmaceutical agent, the polymer fibers are preferably a combination of a biodegradable polymer and a biostable polymer.

Suitable biostable polymers which can be used in the present embodiments include, without limitation, polycarbonate based aliphatic polyurethanes, silicon modificated polyurethanes, polydimethylsiloxane and other silicone rubbers, polyester, polyolefins, polymethyl-methacrylate, vinyl halide polymer and copolymers, polyvinyl aromatics, polyvinyl esters, polyamides, polyimides and polyethers.

Suitable biodegradable polymers which can be used in the present embodiments include, without limitation, poly (L-lactic acid), poly (lactide-co-glycolide), polycaprolactone, polyphosphate ester, poly (hydroxy-butyrate), poly (glycolic acid), poly (DL-lactic acid), poly (amino acid), cyanocrylate, some copolymers and biomolecules such as collagen, DNA, silk, chitozan and cellulose.

Optionally and preferably, graft 40 can also comprise a tubular support structure 75, extending along conduit 48 and/or conduit 50. Tubular support structure 75 can be disposed internally within the conduit(s), or it can be embedded in the walls of the conduits, e.g., between to successive layers Tubular support structure 75 can be any tubular supporting support structure known in the art (to this end see, e.g., WO 02/49535 supra, and U.S. Pat. Nos. 6,945,993, 6,949,120 and 6,939,373). For example, structure 75 can be a deformable mesh of wires made of a metallic material such as, but not limited to, medical grade stainless steel or a material exhibiting temperature-activated shape memory properties, such as Nitinol.

Thus, the present embodiment successfully provides a vascular prosthesis which can be combined with a tubular support structure to create a “stent-graft” assembly, whereby which combines high mechanical strength and self-sealing properties. The advantage of such assembly is that it can be sutured to biological blood vessels while minimizing or preventing leakage due to the suturing procedure.

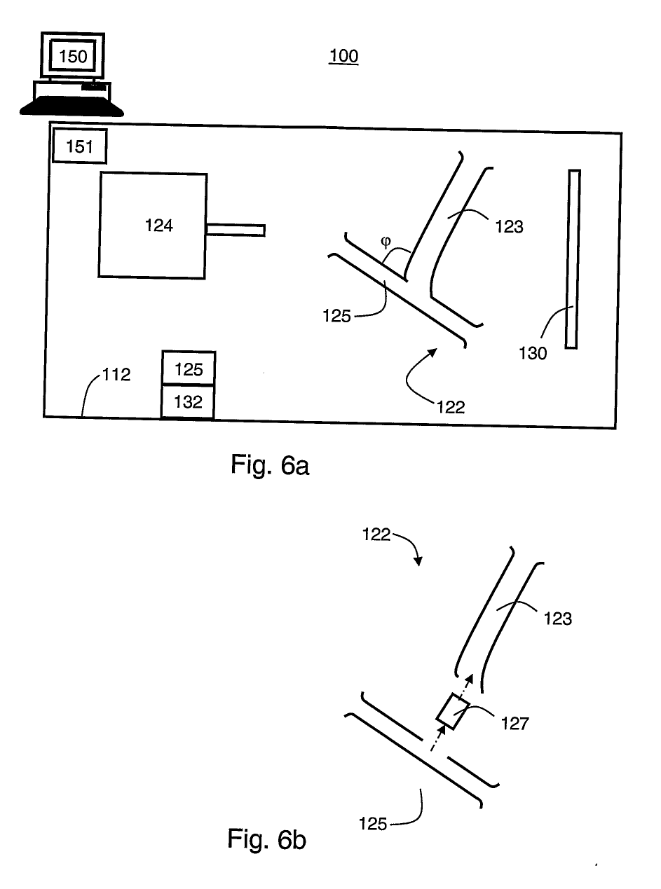

Reference is now made to FIGS. 6a-b, which are schematic illustrations of an apparatus 100 for manufacturing the branching graft of the present embodiments. In its simplest configuration, apparatus 100 comprises a precipitation electrode 122, and a dispenser 124, positioned at a predetermined distance from precipitation electrode 122 and being kept at a first potential relative to precipitation electrode 122.

Precipitation electrode 122 is typically manufactured in accordance with the geometrical properties of the final product which is to be fabricated. In the representative example of FIG. 6, electrode 122 has a T-shape having arms 123 and 125 (arm 123 terminates on the side of arm 125), to enable manufacturing of branching graft having one primary conduit and one secondary conduit branching from the primary conduit. However, this need not necessarily be the case, since, as stated, it may be desired to have a branching graft having more than one secondary conduits. In such cases, precipitation electrode includes more than two arms. One of ordinary skills in the art, provided with the details described herein would know how to adjust precipitation electrode 122 of the present embodiments to include more than two arms (e.g., three) arms. Electrode 122 can be made of, for example, stainless steel, or any other electrically conducting material. The shape of each arm is preferably compatible with the desired shape of the conduit formed thereon. For example, in the embodiment in which the primary conduit has a cross-sectional area which varies, the cross-sectional area of electrode 122 also varies.

The angle φ between arms 123 and 125 is not limited. Preferably, but not obligatorily, φ is an acute angle, e.g., below 70°. Electrode 122 is better illustrated in the explosion diagram of FIG. 6b. According to the presently preferred embodiment of the invention arms 123 and 125 of electrode 122 are detachable. For example, arms 123 and 125 can be connected by a removable end-to-side connector 127. The advantage of making arms 123 and 125 detachable is that such configuration facilitates the post manufacturing removal of the final electrospun product from electrode 122.

Alternatively, arms 123 and 125 can have a permanent connection therebetween, such that electrode 122 remains within the lumens of the final graft. This embodiment is particularly when it is desired to manufacture a graft having a tubular support structure extending between its ports (such as, for example, support 75 hereinabove). Thus, electrode 122 can serve for post manufacture support of the branching graft.

The potential difference between dispenser 124 and precipitation electrode 122 is preferably from about 10 kV to about 100 kV, typically about 60 kV. The potential difference between dispenser 124 and precipitation electrode 22 generate an electric field therebetween.

Dispenser 124 serves for dispensing a liquefied polymer in the electric field to produce polymer fibers precipitating on electrode 122. Precipitation electrode 122 serves for forming the branching graft thereupon.

In accordance with the electrospinning technique, a liquefied polymer is drawn into dispenser 124, and then, subjected to the electric field generated by the potential difference between dispenser 124 and electrode 122, is being charged and dispensed in a direction of electrode 122. Moving with high velocity in the inter-electrode space, jet of liquefied polymer cools or solvent therein evaporates, thus forming fibers which are collected on the surface of electrode 122.

According to a preferred embodiment of the present invention apparatus 100 comprises a subsidiary electrode system 130, which is preferably at a second potential relative to precipitation electrode 122 and configured to shape the aforementioned electric field. A typical potential difference between electrode 122 and electrode system 130 is from about 10 kV to about 100 kV, typically about 50 kV.

Subsidiary electrode system 130 controls the direction and magnitude of the electric field between precipitation electrode 122 and dispenser 124 and as such, can be used to control the orientation of polymer fibers precipitated on electrode 122. In some embodiments, subsidiary electrode system 130 serves as a supplementary screening electrode. Generally, the use of screening results in decreasing the coating precipitation factor, which is particularly important upon cylindrical precipitation electrodes having at least a section of small radii of curvature.

Electrode shapes which can be used in the present embodiments include, but are not limited to, a plane, a cylinder, a torus a rod, a knife, an arc or a ring.

Specifically, a cylindrical or planar subsidiary electrode enables manufacturing intricate-profile products being at least partially with small (from about 0.025 millimeters to about 5 millimeters) radius of curvature. Such subsidiary electrodes are also useful for achieving random or circumferential alignment of the fibers onto precipitation electrode 122.

Electrode system 130 may comprise a plurality of electrodes in any arrangement. The size, shape, position and number of electrodes in system 130 is preferably selected so as to maximize the coating precipitation factor, while minimizing the effect of corona discharge in the area of precipitation electrode 122 and/or so as to provide for controlled fiber orientation upon deposition.

In various exemplary embodiments of the invention system 130 comprises three cylindrical electrodes which can be of different diameters. For example, a large diameter cylindrical electrode can be positioned behind precipitation electrode 122 (with respect to dispenser 124), and two cylindrical electrodes of smaller diameter can be poisoned above and below electrode 122.

The ability to control fiber orientation is important when fabricating vascular prostheses in which a high radial strength and elasticity is important. It will be appreciated that a polar oriented structure can generally be obtained also by wet spinning methods, however in wet spinning methods the fibers are thicker than those used by electrospinning by at least an order of magnitude.

Control over fiber orientation is also advantageous when fabricating composite polymer fiber shells which are manufactured by sequential deposition of several different fiber materials.

Subsidiary electrodes of small radius of curvature, can be used to introduce distortion the electric field in an area adjacent to precipitation electrode 122. For maximal such effect, the diameter of the subsidiary electrode must be considerably smaller than that of precipitation electrode 122, yet large enough to avoid generation of a significant corona discharge.

According to a preferred embodiment of the present invention the position of any electrode of subsidiary electrode system 130 can be varied relative to precipitation electrode 122. Such design further facilitates the ability to control the electric field vector (intensity and direction) near electrode 122.

According to a preferred embodiment of the present invention apparatus 130 further comprises a compartment 112, dispenser 124, electrode 122 and subsidiary electrode system 130. Preferably, but not obligatorily, compartment 112 also encapsulates the power source 125 and circuitry 132 which supply the power to apparatus 100. Compartment 112 is preferably made of a material being transmissive in the visual range. Compartment 112 serves for keeping a clean environment therein. According to a preferred embodiment of the present invention the clean environment is of class 1000 (i.e., less than one thousands particles larger than 0.5 microns in each cubic foot of space) or cleaner. More preferably the clean environment is of class 100 (i.e., less than one thousand particles larger than 0.5 microns in each cubic foot of air space).

More preferably, compartment 112 serves as a climate chamber which besides the clean environment, maintains therein predetermined levels of other environmental conditions such as temperature and humidity.

Thus, according to a preferred embodiment of the present invention the temperature with compartment 112 is kept at a predetermined constant level within an accuracy of ±1° C., more preferably ±0.5° C. even more preferably ±0.2° C., so as to control and maintain the desired evaporation rate during the electrospinning process. Maintenance of accurate temperature within compartment 112 is advantageous because the thickness of the produced polymer fibers and the porosity of the branching graft, depends, inter alia, on the evaporation rate of solvent from the polymer jets emerge from dispenser 124. Preferred temperatures for the operation are from about 22° C. to about 40° C.

Additionally, the humidity within compartment 112 is maintained at a predetermined level to an accuracy of 5% more preferably 3% even more preferably 1%. Maintenance of accurate temperature within compartment 112 is useful for preventing or reducing formation of volume charge. Preferred humidity level, in relative value (the weight or pressure of moisture relative to the maximal weight or pressure of moisture for a given temperature) is about 40%.

Dispenser 124 and/or precipitation electrode 122 preferably rotate such that a relative rotary motion is established between dispenser 124 and electrode 122. Similarly, dispenser 124 and/or electrode 122 preferably move such that a relative linear motion is established between dispenser 124 and electrode 122. For example, in one preferred embodiment, precipitation electrode 122 rotates without performing a linear motion, while dispenser 124 performs a linear motion without performing a rotary motion. In another preferred embodiment, dispenser 124 rotates about electrode 122 and electrode 122 performs a linear reciprocal motion. In an additional preferred embodiment, dispenser 124 performs a spiral motion about electrode 122. The relative motion between dispenser 124 and electrode 122 can be established by any mechanism, such as, but not limited to, an electrical motor, an electromagnetic motor, a pneumatic motor, a hydraulic motor, a mechanical gear and the like.

In various exemplary embodiments of the invention apparatus 100 is controlled by a data processor 150 supplemented by an algorithm for controlling apparatus 100. Data processor 150 can communicate with any of the components of apparatus 100 directly or through a control unit 151 located within compartment 112. The communication can be via communication line or, more preferably, via wireless communication so as to preserve to clean environment in compartment 12. Preferably, but not obligatorily, processor 150 also communicates (e.g., through control unit 151) with source 125 and circuitry 132 for controlling the aforementioned potential differences and for automatically activating and deactivating apparatus 100. According to a preferred embodiment of the present invention processor 150 is configured (e.g., by a suitable computer program) to vary the relative rotary motion and/or relative linear motion between dispenser 124 and electrode 122. As will be appreciated by one ordinarily skilled in the art, different angular and/or linear relative velocities can result in different precipitation rates of polymer fibers on electrode 122. Thus, the computerized control on the motions can be used to select the desired precipitation rate, hence also the desired wall thickness of the branching graft.

Additionally, processor 150 can signal the mechanism for establishing the linear and/or angular motions of dispenser 124 and/or electrode 122 to change the corresponding velocities, at a given instant or instances of the process. This embodiment is particularly useful when manufacturing multilayer structures. Thus, by selecting different motion characteristics of dispenser 124 and/or electrode 122 for different layers, the electrospinning process for each layer is at a different precipitation rate, resulting in a different density of fibers on the formed layer. Since the porosity of the layer depends on the density of fiber, such process can be used for manufacturing multilayer branching grafts in which the layers have predetermined and different porosities. Additionally, each layer can have a different wall thickness, which can also be controlled as further detailed above.

In various exemplary embodiments of the invention, the branching graft is to manufactured as follows.

One or more liquefied polymers are provided and introduced into the dispenser. The liquefied polymer(s) can also be mixed with one or more conductivity control agents or charge control agents for improving the interaction of the fibers with the electric field. The distance between the precipitation electrode and the subsidiary electrodes, the distance between the dispenser and the precipitation electrode, and the angle between the dispenser and the precipitation electrode are adjusted by the adjustments mechanism and recorded into the data processor.

The dispenser, precipitation electrode and subsidiary electrode system are sealed by the compartment and the appropriate environmental conditions are established.

Parameters, such as, but not limited to, wall thickness, number of layer, angular and linear velocities, temperature, hydrostatic pressure, polymer viscosities, and the like, are recorded into the data processor which Also recorded are the types of polymers.

Apparatus 100 is activated and the liquefied polymer is extruded under the action of the hydrostatic pressure through the spinnerets. As soon as meniscus of the extruded liquefied polymer forms, a process of solvent evaporation or cooling starts, which is accompanied by the creation of capsules with a semi-rigid envelope or crust. Because the liquefied polymer possesses a certain degree of electrical conductivity, the capsules become charged by the electric field. Electric forces of repulsion within the capsules lead to a drastic increase in hydrostatic pressure. The semi-rigid envelopes are stretched, and a number of point micro-ruptures are formed on the surface of each envelope leading to spraying of ultra-thin jets of the liquefied polymer from the spinnerets.

Under the effect of a Coulomb force, the jets depart from the dispenser and travel towards the opposite polarity electrode, i.e., the precipitation electrode. Moving with high velocity in the inter-electrode space, the jet cools or solvent therein evaporates, thus forming fibers which are collected on the surface of the precipitation electrode.

Once a first layer is formed, the data processor signals the dispenser to reselect a different liquefied polymer (in embodiments in which different liquefied polymers are used for different layers), and the motion mechanisms to change the rotary and/or linear velocities (in embodiments in which different the layers have different wall thicknesses and/or different porosities). The signaling of the data processor is preferably performed without ceasing the electrospinning process, such that the new layer is formed substantially immediately after the previous layer.

Once all the layers are formed, the compartment is opened and the precipitation electrode, including the branching graft formed thereupon is disengaged from the system. The branching graft is then removed from the precipitation electrode.

According to a preferred embodiment of the present invention the removal of the branching graft is performed as follows. The precipitation electrode, including the branching graft is irradiated by ultrasound radiation. It was found by the inventor of the present invention that ultrasound radiation facilitates the removal of the branching graft from the electrode. Additionally and more preferably, the precipitation electrode including the branching graft can also be subjected to at least one substantially abrupt temperature change. The abrupt temperature change can be applied by any suitable heat carrier, including, without limitation, a liquid bath. The removal process can also be controlled by the data processor. Specifically, the data processor can control the duration and level of the applied temperatures and/or the ultrasound radiation.

The precipitation electrode including the branching graft is immersed in an ultrasonic bath of low temperature (about 0° C.) for a first predetermined period (about 1-10 minutes, more preferably 3-5 minutes). Subsequently, the precipitation electrode including the branching graft is immersed in another ultrasonic bath of high temperature (from about 40° C. to about 100° C.) for a second predetermined period (about 1-10 minutes, more preferably 3-5 minutes). According to a preferred embodiment of the present invention once the above thermal treatment is completed the arms of the precipitation electrode are detached (preferably while the branching graft is on the precipitation electrode). In an alternative embodiment, the detachment of the arms can precede the thermal treatment. Irrespectively, each arm of the precipitation electrode is separately pulled out from the branching graft.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A method of bypassing a coronary artery being at least partially occluded, comprising: using a branching graft for establishing direct fluid communication between an upstream vascular location being upstream an occlusion in the coronary artery, a downstream vascular location being downstream said occlusion, and a distal vascular location, wherein said distal vascular location is selected on a distal artery or a distal portion of an artery to ensure that arterial blood flow in said distal artery or said distal portion of said artery generates sufficient pressure gradient in said branching graft to maintain said direct fluid communication.

2. The method of claim 1, further comprising establishing direct fluid communication between: at least one additional downstream vascular location, said upstream vascular location, said downstream vascular location and said distal vascular location.

3. An artificial branching graft for implantation in body vasculature during a coronary artery bypass procedure, comprising: a primary conduit, at least one secondary conduit branching from said primary conduit, and at least one unidirectional valve designed and constructed to ensure unidirectional flow within at least a portion of said primary conduit.

4. The artificial branching graft of claim 3, wherein at least a portion of said primary conduit has a generally oval cross-sectional shape.

5. The artificial branching graft of claim 3, wherein at least one end of said primary conduit is bent with respect to a longitudinal axis of said primary conduit.

6. The artificial branching graft of claim 5, wherein said bending of said primary conduit is characterized by an acute angle measured at a convex side of said bending.

7. A method of bypassing a coronary artery being at least partially occluded, comprising: using the artificial implantable branching graft of claim 3 for establishing direct fluid communication between an upstream vascular location upstream an occlusion in the coronary artery, a downstream vascular location downstream said occlusion, and a distal vascular location, wherein said distal vascular location is selected on a distal artery or a distal portion of an artery to ensure that arterial blood flow in said distal artery or said distal portion of said artery generates sufficient pressure gradient in said branching graft to maintain said direct fluid communication.

8. The method of claim 1, wherein said branching graft comprises at least one harvested blood vessel.

9. The method of claim 1, wherein said branching graft comprises an artificial graft.

10. The method of claim 1, wherein the primary conduit of said branching graft is connected to said distal vascular location at an acute angle defined relative to said arterial blood flow.

11. The method or branching graft of claim 6, wherein said acute angle is smaller or equals 70 degrees.

12. The method or branching graft of claim 1, wherein the primary conduit of said branching graft is characterized by a varying cross-sectional area.

13. The method or branching graft of claim 12, wherein said varying cross-sectional area varies in a non-monotonic manner.

14. The method or branching graft of claim 12, wherein said varying cross-sectional area varies in a monotonic manner.

15. The method of claim 12, wherein said varying cross-sectional area is larger at said upstream vascular location than at said distal vascular location.

16. The method of claim 12, wherein said varying cross-sectional area has a minimal value at a location on said primary conduit being other than said upstream vascular location and said distal vascular location.

17. The method or branching graft of claim 12, wherein said varying cross-sectional area has a minimal value at location on said primary conduit being other than the ends of said primary conduit.

18. The method of claim 1, wherein said distal vascular location is on the aorta.

19. The method of claim 18, wherein said distal vascular location is on the descending aorta.

20. The method of claim 18, wherein said distal vascular location is on the aortic arch.

21. The method of claim 1, wherein said distal vascular location is on an aortic branch.

22. The method of claim 21, wherein said distal vascular location is on the brachiocephalic artery.

23. The method of claim 21, wherein said distal vascular location is on the left carotid artery.

24. The method of claim 21, wherein said distal vascular location is on the left subclavian artery.

25. The method of claim 21, wherein said upstream vascular location is on the ascending aorta.

26. The method of claim 21, wherein said upstream vascular location is on the coronary artery.

27. The method of claim 21, wherein said upstream vascular location is on an aortic branch.

28. The method of claim 21, wherein said downstream vascular location is on the coronary artery.

29. The method of claim 21, wherein said downstream vascular location is on a branch of the coronary artery.

30. The branching graft or method of claim 3, wherein at least one of said primary conduit and said at least one secondary conduit is a tubular structure of non-woven polymer fibers.

31. The branching graft or method of claim 3, wherein the branching graft comprises at least one generally annular flexible support structure supporting at least one end of said primary conduit and/or said at least one secondary conduit.

32. The branching graft or method of claim 31, wherein said support structure is an embedded support structure.

33. The branching graft or method of claim 3, wherein the branching graft further comprises a tubular support structure extending along at least one of said primary conduit and/or said at least one secondary conduit.

34. The branching graft or method of claim 33, wherein said tubular support structure is embedded in the respective conduit.

35. The branching graft or method of claim 3, wherein at least one of said primary conduit and/or said at least one secondary conduit comprises a plurality of layers each layer of said plurality of layers being made of non-woven polymer fibers.

36. The branching graft or method of claim 30, wherein at least one of said primary conduit and/or said at least one secondary conduit includes a pharmaceutical agent incorporated therein for delivery of said pharmaceutical agent into the body vasculature during or after implantation of the branching graft within said body vasculature.
